(19) United States
(12) Patent Application Publication
(10) Pub. No.: US 2025/0073372 A1
(43) Pub. Date: Mar. 6, 2025

(54) SURFACTANT COMPOSITION INCLUDING AMPHIPHILIC BLOCK COPOLYMER BASED ON POLYHYDROXYALKANOATE

(71) Applicant: POSTECH Research and Business Development Foundation, Pohang-si (KR)

(72) Inventors: Dong Soo HWANG, Pohang-si (KR); Gi-Ra Yl, Pohang-si (KR); Geonho SONG, Pohang-si (KR); Yunhee JEONG, Pohang-si (KR)

(73) Assignee: POSTECH Research and Business Development Foundation, Pohang-si (KR)

(21) Appl. No.: 18/825,465

(22) Filed: Sep. 5, 2024

(30) Foreign Application Priority Data
Sep. 5, 2023 (KR) 10-2023-0117800
Sep. 3, 2024 (KR) 10-2024-0119224

(51) Int. Cl. A61L 24/04; A61K 8/90; A61L 24/00; A61Q 19/00; C08G 63/06; C08G 81/00

(52) U.S. Cl. CPC A61L 24/046; A61K 8/90; A61L 24/0042; A61Q 19/00; C08G 63/06; C08G 81/00; A61K 2800/10

(57) ABSTRACT

The present disclosure relates to a surfactant composition including an amphiphilic block copolymer based on polyhydroxyalkanoate (PHA), the surfactant composition is highly safe for the human body and the environment due to its excellent biodegradability and biocompatibility, and a hemostatic agent and medical adhesive including the same are effective in hemostasis, wound healing, tissue adhesion, and inhibition of bacterial infection.

### CROSS-REFERENCE TO RELATED APPLICATIONS

This application claims priority under 35 U.S.C. § 119 to Korean Patent Application No. 10-2024-0119224, filed on 2024 Sep. 3 and No. 10-2023-0117800, filed on 2023 Sep. 5, in the Korean Intellectual Property Office, the disclosure of which is incorporated herein by reference in its entirety.

### TECHNICAL FIELD

The present disclosure relates to a surfactant composition including an amphiphilic block copolymer based on polyhydroxyalkanoate, and a medical adhesive and cosmetic composition including the same.

### BACKGROUND

A surfactant plays a very important role in the modern medical and cosmetic industries. The surfactant significantly improves the quality and performance of products such as cosmetics, medical products, and detergents through its functions such as cleaning, emulsifying, and dispersing. In terms of the cleaning function, the surfactant removes contaminants, and in terms of the emulsifying function, the surfactant stably combines immiscible substances such as water and oil. Through the dispersing function, the surfactant helps maintain product stability by evenly distributing particles.

However, the existing synthetic surfactants have several problems in terms of environmental sustainability and safety for a human body. A large number of synthetic surfactants are not biodegradable and therefore accumulate in the environment, causing pollution. These substances are difficult to degrade in the natural environment, which may cause problems such as water pollution and soil pollution. For example, the synthetic surfactants may cause water pollution when they accumulate in aquatic ecosystems, and may cause soil pollution when they accumulate in soil.

In addition, the synthetic surfactants that are not degraded in the body may act as potential toxic substances. These substances that are absorbed into the body may accumulate over a long period of time, which may have negative health effects. For example, some synthetic surfactants accumulate in the body, which may have negative effects on the liver, kidney, and the like and may also cause an immune response. For these reasons, the medical and cosmetic industries require the development of alternative surfactants having biocompatibility and biodegradability.

### SUMMARY

An embodiment of the present disclosure is directed to providing a surfactant composition that is degradable in vivo and in vitro and therefore is environmentally friendly.

Another embodiment of the present disclosure is directed to providing a surfactant composition that may minimize an immune response caused by absorption of a surfactant into the body and may implement wound healing, inhibition of infection, and antibacterial effects.

In one general aspect, a surfactant composition includes an amphiphilic block copolymer including: a hydrophobic block containing polyhydroxyalkanoate (PHA); and a hydrophilic block.

The polyhydroxyalkanoate may contain a repeating unit derived from 3-hydroxybutyrate and/or a repeating unit derived from 4-hydroxybutyrate.

A content of the repeating unit derived from 4-hydroxybutyrate may be 0.1 to 60 wt % with respect to the total weight of the polyhydroxyalkanoate.

The polyhydroxyalkanoate may be a poly (3-hydroxybutyrate-co-4-hydroxybutyrate) copolymer.

The hydrophilic block may include at least one selected from polyethylene glycol (PEG), polyglycerol (PG), hyaluronic acid, dextran, and mannan.

The amphiphilic block copolymer may be produced by treating a hydrophilic substance constituting the hydrophilic block with a peroxide and then mixing the hydrophilic substance with polyhydroxyalkanoate.

A weight ratio of the hydrophobic block and the hydrophilic block may be 3,000:1 to 1:10.

A number average molecular weight of the hydrophilic block may be 1,000 to 40,000 g/mol.

A molecular weight of the polyhydroxyalkanoate may be 10,000 to 1,200,000 g/mol.

A concentration of the amphiphilic block copolymer may be 0.1 to 75% (w/v).

In another general aspect, a hemostatic agent includes the surfactant composition according to an exemplary embodiment of the present disclosure.

In still another general aspect, a medical adhesive includes the surfactant composition according to an exemplary embodiment of the present disclosure.

In still another general aspect, a cosmetic composition includes the surfactant composition according to an exemplary embodiment of the present disclosure.

Other features and aspects will be apparent from the following detailed description, the drawings, and the claims.

### BRIEF DESCRIPTION OF THE DRAWINGS

FIG. 1 is a schematic view illustrating a preparation process of PHA (L6)-co-PEG.

A of FIG. 2 illustrates a result of measuring interfacial energy at an interface between water and chloroform, and B of FIG. 2 illustrates stability of an emulsion observed with an optical microscope.

A and B of FIG. 3 illustrate results of measuring a size distribution and a polydispersity index (PDI) of particles passing through a microfludizer according to the number of times.

FIG. 4 is a schematic view illustrating a preparation process of PHA (L6)-co-Dextran.

FIG. 5 illustrates a result of FT-IR analysis of PHA (L6)-co-Dextran. A and B of FIG. 6 illustrate wound healing processes over time in terms of appearance, C of FIG. 6 illustrates histological processes, and D of FIG. 6 illustrates a quantitative evaluation result of a wound width, when each sample is treated.

### DETAILED DESCRIPTION OF EMBODIMENTS

Hereinafter, the present disclosure will be described in detail. Unless otherwise defined, the terms used in the present specification should be interpreted as generally understood by those skilled in the art to which the present disclosure pertains. The drawings and exemplary embodiments of the present specification are for those skilled in the art to easily understand and implement the present disclosure, the contents that may obscure the gist of the present disclosure in the drawings and exemplary embodiments may be omitted, and the present disclosure is not limited to the drawings and exemplary embodiments.

Unless the context clearly indicates otherwise, singular forms used in the present specification may be intended to include plural forms.

In addition, a numerical range used in the present specification includes upper and lower limits and all values within these limits, increments logically derived from a form and span of a defined range, all double limited values, and all possible combinations of the upper and lower limits in the numerical range defined in different forms. Unless otherwise specifically defined in the specification of the present disclosure, values out of the numerical range that may occur due to experimental errors or rounded values also fall within the defined numerical range.

The terms “comprise(s)”, “include(s)”, “have (has)”, and the like used in the present specification indicate the presence of features or components described in the specification, and do not preclude the addition of one or more other features or components, unless specifically limited.

The present disclosure provides a surfactant composition including an amphiphilic block copolymer including: a hydrophobic block containing polyhydroxyalkanoate (PHA); and a hydrophilic block. The surfactant composition may bond biological tissue and may exhibit a hemostatic effect, a wound healing effect, a bacterial infection inhibiting effect, an antibacterial effect, and the like for biological tissue.

The surfactant composition may be a composition in which the amphiphilic block copolymer is dispersed in a common solvent or solution. Specifically, the solvent or solution is not particularly limited, and specific examples thereof include purified water, a saline solution, or a PBS buffer solution. Specifically, the amphiphilic block copolymer included in the surfactant composition may be 0.01 to 5 wt % with respect to the total weight of the composition, but is not limited thereto.

The amphiphilic block copolymer included in the surfactant composition may be an A-B type diblock copolymer, an A-B-A type triblock copolymer, or an A-B-A-B type tetrablock copolymer, wherein A and B may be hydrophobic blocks or hydrophilic blocks, but are not limited thereto.

Specifically, the amphiphilic block copolymer may be a diblock copolymer with the link of [hydrophobic block]−[hydrophilic block], and may be a triblock copolymer with the link of [hydrophobic block]−[hydrophilic block]−[hydrophobic block] or [hydrophilic block]−[hydrophobic block]−[hydrophilic block].

In addition, according to an exemplary embodiment, a weight ratio of the hydrophobic block and the hydrophilic block in the amphiphilic block copolymer may be 3,000:1 to 1:10, and specifically, may be 2, 500:1 to 1:10, but is not limited thereto.

The hydrophilic block may include at least one selected from polyethylene glycol (PEG), polyglycerol (PG), polyvinyl alcohol (PVA), polyacrylamide (PAM), hydroxypropyl methylcellulose (HPMC), polyvinylpyrrolidone (PVP), carboxymethyl cellulose (CMC), polyglutamic acid (PGA), levan, starch, amylose, amylo-pectin, cellulose, hyaluronic acid, dextran, and mannan, and specifically, may be at least one selected from polyethylene glycol (PEG), levan, starch, amylose, amylo-pectin, cellulose, hyaluronic acid, dextran, and mannan. According to an exemplary embodiment, the hydrophilic block may include at least one selected from polyethylene glycol (PEG), hyaluronic acid, dextran, and mannan.

A number average molecular weight of the hydrophilic block may be 1,000 to 20,000 g/mol, 2,000 to 15,000 g/mol, 3,000 to 10,000 g/mol, or 4,000 to 8,000 g/mol, and specifically, may be 4,000 to 5,000 g/mol.

The polyhydroxyalkanoate (PHA) may contain a repeating unit derived from one or more selected from the group consisting of 3-hydroxybutyrate (3HB), 4-hydroxybutyrate (4HB), 3-hydroxypropionate (3HP), 3-hydroxyhexanoate (3HH), 3-hydroxyvalerate (3HV), 4-hydroxyvalerate (4HV), 5-hydroxyvalerate (5HV), and 6-hydroxyhexanoate (6HH).

According to an exemplary embodiment, the polyhydroxyalkanoate (PHA) may contain a repeating unit derived from 3-hydroxybutyrate (3HB) and/or a repeating unit derived from 4-hydroxybutyrate (4HB).

A content of the repeating unit derived from 4-hydroxybutyrate may be 0.1 to 60 wt %, and specifically, 1 to 48 wt %, with respect to the total weight of the polyhydroxyalkanoate (PHA).

According to an exemplary embodiment, the polyhydroxyalkanoate (PHA) may be a poly (3-hydroxybutyrate-co-4-hydroxybutyrate) copolymer.

According to an exemplary embodiment, the amphiphilic block copolymer may be produced by treating a hydrophilic substance constituting the hydrophilic block with a peroxide and then mixing the hydrophilic substance with polyhydroxyalkanoate (PHA). Specifically, a copolymer may be formed by treating the hydrophilic substance with a peroxide to introduce an aldehyde group (—CHO), and then reacting the generated aldehyde group with polyhydroxyalkanoate (PHA).

Specifically, the peroxide may be sodium peroxide (NaIO₄), hydrogen peroxide (H₂O₂), potassium persulfate (K₂S₂O₈), or ammonium persulfate ((NH₄)₂S₂O₈), and more specifically, may be sodium peroxide (NaIO₄).

A molecular weight (weight average molecular weight) of the polyhydroxyalkanoate may be 10,000 to 1, 200, 000 g/mol, and specifically, may be 20,000 to 1,100,000 g/mol, 30,000 to 1,000,000 g/mol, 40,000 to 900,000 g/mol, 45,000 to 850,000 g/mol, 50,000 to 800,000 g/mol, 60,000 to 750,000 g/mol, 70,000 to 700,000 g/mol, 80,000 to 600,000 g/mol, 90,000 to 500,000 g/mol, 100,000 to 400,000 g/mol, 200,000 to 800,000 g/mol, or 300,000 to 700,000 g/mol. As the molecular weight of the polyhydroxyalkanoate (PHA) is within the above range, the adhesive performance to biological tissue may be improved.

A concentration of the amphiphilic block copolymer in the surfactant composition of the present disclosure may be 0.1 to 75% (w/v), and specifically, may be 0.2 to 70% (w/v), 0.2 to 60% (w/v), 0.2 to 50% (w/v), 0.3 to 40% (w/v), 0.3 to 30% (w/v), 0.3 to 25% (w/v), 0.3 to 16% (w/v), 0.35 to 15.6% (w/v), 0.7 to 13% (w/v), 3 to 11% (w/v), or 6 to 9% (w/v). As the concentration of the amphiphilic block copolymer in the surfactant composition is within the above range, the hemostatic effect, wound healing effect, and adhesive performance of the surfactant composition of the present disclosure may be significantly improved.

The present disclosure provides a hemostatic agent including the surfactant composition according to an exemplary embodiment of the present disclosure. The hemostatic agent has excellent biocompatibility may and effectively perform hemostasis because it has low immunogenicity. The surfactant composition of the present disclosure may efficiently perform hemostasis of injured biological tissue by including the amphiphilic block copolymer.

The hemostatic agent may be formulated into various forms. Specifically, the hemostatic agent according to an exemplary embodiment of the present disclosure may have a formulation such as a powder form, a liquid form, a gel form, or a sheet form (film form).

The present disclosure provides a medical adhesive including the surfactant composition according to an exemplary embodiment of the present disclosure. The medical adhesive may have a tissue adhesive strength of 15 kPa or more as measured at 100% relative humidity and room temperature (for example, 20 +5° C., specifically 25° C.). Specifically, the medical adhesive may have an adhesive strength to biological tissue of 1 to 50kPa, 2 to 50 kPa, 3 to 40 kPa, or 5 to 30 kPa. As the adhesive strength to biological tissue is within the above range, the medical adhesive may exhibit high adhesive performance not only for biological tissue without moisture but also for biological tissue with moisture (for example, tissue with a wet surface or tissue in water).

The medical adhesive according to an exemplary embodiment of the present disclosure may further include a biocompatible polymer and/or an adhesive material other than the surfactant composition including the amphiphilic block copolymer. Specifically, the biocompatible polymer may be catechol, caffeic acid, gallic acid, or tannin; a polysaccharide-based polymer such as chitosan, hyaluronic acid, alginic acid, or dextran; or a protein-based polymer such as collagen or gelatin. The adhesive material may be a protamine, a mussel adhesive protein, or a suckerin.

In addition, the medical adhesive may further include a commonly known pharmacologically active substance. Specifically, the pharmacologically active substance may be an analgesic, an anti-inflammatory agent, an antibacterial agent, or an antifungal agent.

The medical adhesive may be formulated into various forms. Specifically, the medical adhesive according to an exemplary embodiment of the present disclosure may have a formulation such as a powder form, a liquid form, a gel form, or a sheet form (film form).

The present disclosure provides a cosmetic composition including the surfactant composition according to an exemplary embodiment of the present disclosure.

The surfactant composition may be included in an amount of 0.5 to 50 wt %, and specifically, 1.0 to 55 wt %, 1.5 to 45 wt %, 2.0 to 30 wt %, 2.5 to 25 wt %, or 3.0 to 20 wt %, with respect to the total weight of the cosmetic composition.

Hereinafter, the surfactant composition according to the present disclosure will be described in more detail with reference to specific examples. However, the following examples are only reference examples for describing the present disclosure in detail, and the present disclosure is not limited thereto and may be implemented in various forms. In addition, the terms used in the present disclosure are only to effectively describe specific examples, and are not intended to limit the present disclosure.

### EXAMPLE 1

### Preparation of PHA (L6)-co-PEG

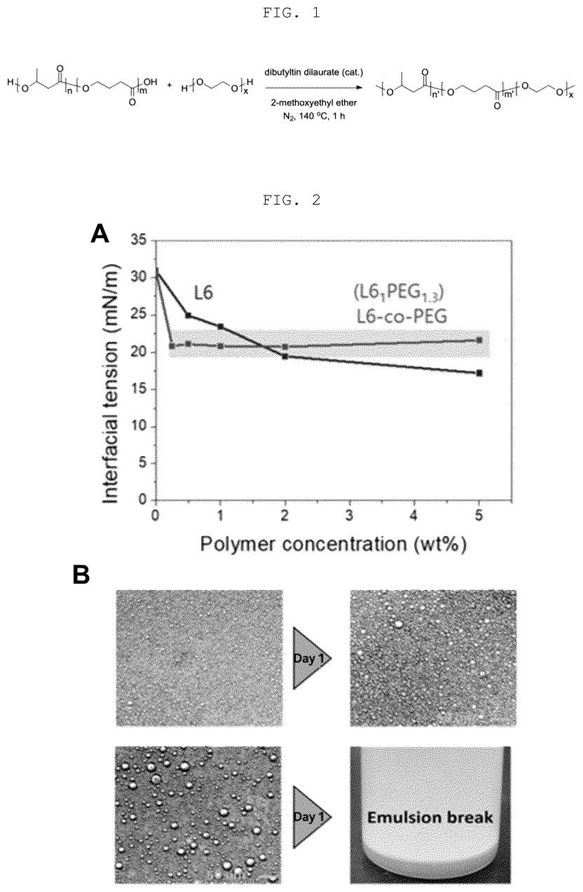

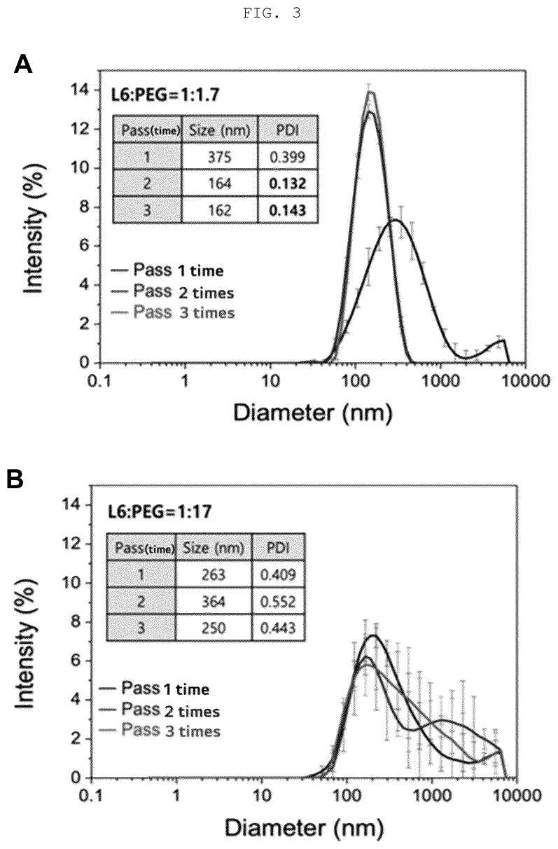

As shown in the reaction formula in FIG. 1, a copolymer, in which PHA (L6: a content of 4-hydroxybutyrate (4HB) was 6% with respect to 3-hydroxybutyrate (3HB)) was used as a hydrophobic substance and polyethylene glycol (PEG) was used as a hydrophilic substance, was synthesized.

Specifically, PHA (L6) and polyethylene glycol (4 kDa) were quantified at a molar ratio of 1:1.7 or 1:17, and a mixture was prepared so that a weight ratio of the total solid reactant and 2-methoxyethyl ether, which is a solvent, was 1:5. Thereafter, the mixture was stirred at 140°° C. in a nitrogen atmosphere for about 20 minutes. Dibutyltin dilaurate, which is a reaction catalyst, was prepared with 1 mL of a solvent and then added to the mixture under stirring at a concentration of 0.79 mM, and the reaction was continued for 1 hour. Thereafter, a washing process of pouring the reactant into cold water to form a precipitate, performing ultrasonication at 50°° C., and filtering the precipitate was performed 2 or 3 times, and a final product was dried under vacuum.

### EXAMPLE 2

### Preparation of PHA-co-PEG

The same process as in Example 1 was performed, except that the composition of PHA was 3-hydroxybutyrate (3HB) alone.

### COMPARATIVE EXAMPLE 1

PHA (L6: the content of 4-hydroxybutyrate (4HB) was 6% with respect to 3-hydroxybutyrate (3HB)) of Example 1 was used in Comparative Example 1.

### Interfacial Energy

The results of measuring the interfacial energy at the interface between water and chloroform of Example 1 and Comparative Example 1 are illustrated in A and B of FIG. 2.

According to A of FIG. 2, in Comparative Example 1, it was confirmed that the interfacial energy continuously decreased as the concentration of the polymer increased, whereas in Example 1, it was confirmed that, when the concentration of the copolymer was 0.25 wt %, the interfacial energy was maintained at a certain level, and accordingly, the critical micelle point (CMC) was formed at a concentration of 0.25 wt %.

In addition, as illustrated in B of FIG. 2, unlike Comparative Example 1, in Example 1, it was confirmed that the emulsion was stable even after one day. Accordingly, in Example 1, it was considered that the copolymer effectively acted as a surfactant that stabilizes the interface of the emulsion compared to Comparative Example 1 in which no copolymer was formed.

### Emulsion Stability

In Example 1, the emulsion stability when the molar ratio of PHA (L6) and polyethylene glycol (4 kDa) was 1:1.7 or 1:17 was confirmed. Specifically, in the experiment, an emulsion was formed using only the copolymer of Example 1 and distilled water and then particles were formed by evaporating chloroform using a microfluidizer. The results of measuring the size distribution and polydispersity index (PDI) of the particles according to the number of times the particles passed through the microfluidizer are illustrated in A and B of FIG. 3.

According to A and B of FIG. 3, when the molar ratio of PHA (L6) and PEG was 1:1.7, it was confirmed that as the number of times the particles passed through the microfluidizer increased, the size of the particles decreased and the distribution became more homogeneous. On the other hand, when the molar ratio of PHA (L6) and PEG was 1:17, it was confirmed that the size and distribution of the particles were imbalanced regardless of the number of times the particles passed through the microfluidizer.

### EXAMPLE 3

### Preparation of PHA (L6)-co-Dextran

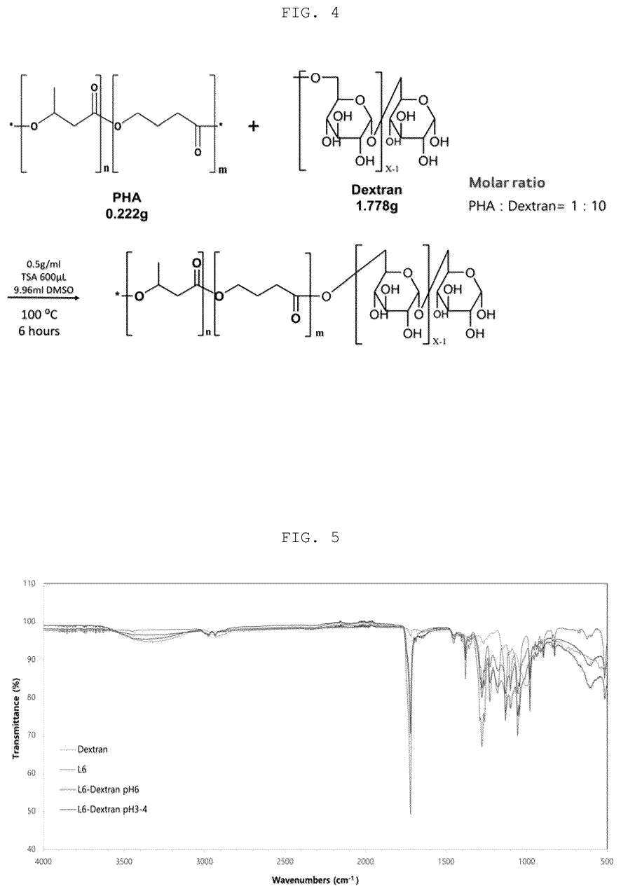

As shown in the reaction formula in FIG. 4, the same process as in Example 1 was performed, except that the hydrophilic substance was changed to dextran.

Specifically, PHA powder (L6, 50 kDa) and dextran (from Leuconostoc spp., 40 kDa) were quantified at a molar ratio of 1:10, and a mixture was prepared so that a weight ratio of a solid reactant and dimethyl sulfoxide, which is a solvent, was about 1:5. The mixture was stirred at 100° C. for about 1 hour. After confirming that the solid reactant was partially dissolved in the solvent, a reflux cooler was connected. In an argon atmosphere, p-toluenesulfonic acid, which is a reaction catalyst, was prepared with 1 mL of a solvent, and then the reactant connected to the device was added to the mixture under stirring at a concentration of 149.4 mM, and the reaction was continued for 4 hours. Thereafter, 1 g of magnesium sulfate was added, and then, the reaction was continued for 2 hours. The reactant was filtered under reduced pressure, the obtained solid product was washed with distilled water, the obtained liquid product was poured into cold water to precipitate, and the obtained final product was dried under vacuum.

### FT-IR Analysis

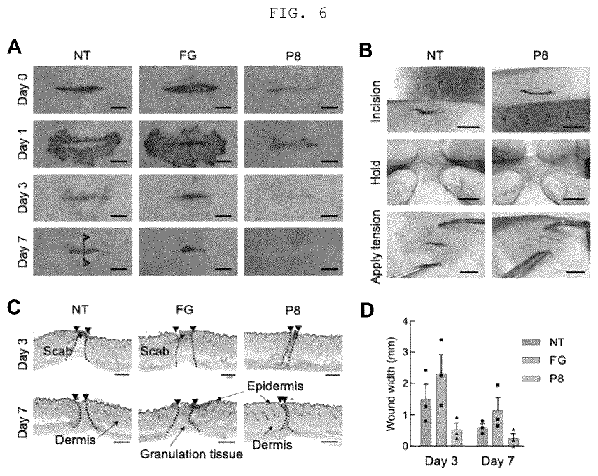

The FT-IR analysis result of PHA (L6)-co-Dextran synthesized through Example 3 is illustrated in FIG. 5. According to FIG. 5, it was considered that a copolymer of PHA and dextran was formed because it was confirmed that dextran was bound to the PHA polymer and a polysaccharide pattern appeared together with a PHA pattern at around 3, 300 cm⁻¹ and 980 cm⁻¹.

### EXPERIMENTAL EXAMPLE 1

### Tissue Adhesion and Wound Healing Effect

A wound of about 2 cm was made on the back skin of a mouse, and the adhesive effect on the wound when the wound was bonded with each of the copolymer dispersion (P8) of Example 1 and a bioadhesive material (fibrin glue, FG) and when no treatment (NT) was performed is illustrated in A to D of FIG. 6.

In summary, it was confirmed through A to C of FIG. 6 that, in Example 1, the wound site healed up more completely than with FG and NT, and in particular, it was confirmed through D of FIG. 6 that the wound area was effectively reduced.

As set forth above, the surfactant composition according to an exemplary embodiment of the present disclosure has excellent biodegradability and biocompatibility, and is therefore highly safe for the human body and the environment.

The surfactant composition according to an exemplary embodiment of the present disclosure is effective in hemostasis, wound healing, tissue adhesion, and inhibition of bacterial infection.

Hereinabove, although the present disclosure has been described by specific matters and limited examples and comparative examples, they have been provided only for assisting in the entire understanding of the present disclosure. Therefore, the present disclosure is not limited to the examples. Various modifications and changes may be made by those skilled in the art to which the present disclosure pertains from this description.

Therefore, the spirit of the present disclosure should not be limited to the described exemplary embodiments, but the claims and all modifications equal or equivalent to the claims are intended to fall within the spirit of the present disclosure.

What is claimed is:

1. A surfactant composition comprising:
an amphiphilic block copolymer including:
a hydrophobic block containing polyhydroxyalkanoate (PHA); and
a hydrophilic block.

2. The surfactant composition of claim 1, wherein the polyhydroxyalkanoate contains a repeating unit derived from 3-hydroxybutyrate and/or a repeating unit derived from 4-hydroxybutyrate.

3. The surfactant composition of claim 2, wherein a content of the repeating unit derived from 4-hydroxybutyrate is 0.1 to 60 wt % with respect to the total weight of the polyhydroxyalkanoate.

4. The surfactant composition of claim 1, wherein the polyhydroxyalkanoate is a poly (3-hydroxybutyrate-co-4-hydroxybutyrate) copolymer.

5. The surfactant composition of claim 1, wherein the hydrophilic block includes at least one selected from polyglycerol (PG), polyethylene glycol (PEG), hyaluronic acid, dextran, and mannan.

6. The surfactant composition of claim 1, wherein the amphiphilic block copolymer is produced by treating a hydrophilic substance constituting the hydrophilic block with a peroxide and then mixing the hydrophilic substance with polyhydroxyalkanoate.

7. The surfactant composition of claim 1, wherein a weight ratio of the hydrophobic block and the hydrophilic block is 3,000:1 to 1:10.

8. The surfactant composition of claim 1, wherein a number average molecular weight of the hydrophilic block is 1,000 to 40,000 g/mol.

9. The surfactant composition of claim 1, wherein a molecular weight of the polyhydroxyalkanoate is 10,000 to 1,200,000 g/mol.

10. The surfactant composition of claim 1, wherein a concentration of the amphiphilic block copolymer is 0.1 to 75% (w/v).

11. A hemostatic agent comprising the surfactant composition of claim 1.

12. A medical adhesive comprising the surfactant composition of claim 1.

13. A cosmetic composition comprising the surfactant composition of claim 1.
